(19) United States
(12) Patent Application Publication
(10) Pub. No.: US 2021/0106535 A1
(43) Pub. Date: Apr. 15, 2021

(54) NATURAL AND/OR ORGANIC DISINTEGRANT PRE-MIXES FOR (ORAL) SOLID DOSAGE FORMS

(71) Applicants: Bonutra AG, Steinhausen (CH); Herbitat Lifesciences, LLP, Mumbai (IN)

(72) Inventors: Ritesh Patel, Somerset, NJ (US); Somsuvra Ghatak, Somerset, NJ (US); Felix Specht, Shelbyville, KY (US); Mahmud Yunis, Wiesbaden (DE)

(73) Assignees: Bonutra AG, Steinhausen (CH); Herbitat Lifesciences, LLP, Mumbai (IN)

(21) Appl. No.: 17/069,171

(22) Filed: Oct. 13, 2020

(30) Foreign Application Priority Data
Oct. 14, 2019 (EP) 19202932.0

(51) Int. Cl. A61K 9/20

(52) U.S. Cl. CPC A61K 9/2068; A61K 9/2054; A61K 9/205

(57) ABSTRACT

Natural disintegrant pre-mixes which are useful for formulating solid dosage forms for oral administration of drugs, vitamins, food supplements, etc. The composition comprises a blend about 20% to about 90% by weight of Psyllium Husk and about 5% to about 60% by weight of Oat Fiber (all weight percentages being based on the dry weight of the disintegrant composition). The composition may further comprise about 1% to about 25% by weight of at least one further component selected from guar gum, Locust bean gum, gum karaya, Aegle marmelose gum, Mangifera indica gum, and a combination thereof and/or about 1% to about 20% by weight of at least one further component selected Apple Fiber, Wheat Fiber, Pea Fiber, Cellulose Powder, Agave Powder, Bamboo Fiber, and a combination thereof (all weight percentages being based on the dry weight of the disintegrant composition).

### CROSS-REFERENCE TO RELATED APPLICATION

The present application claims priority to European Patent Application No. 19202932.0, filed Oct. 14, 2019, which is hereby incorporated herein by reference in its entirety.

### BACKGROUND OF THE INVENTION

### Field of the Invention

The invention relates to the field of natural disintegrant pre-mixes which are useful for formulating solid dosage forms for oral administration of drugs, vitamins, food supplements, etc. The pre-mixes promote the break-up of the tablet (and capsule “slugs”) into smaller fragments in an aqueous environment thereby increasing the available surface area and promoting the release of the active nutraceutical/pharmaceutical substance.

### Description of Related Art

In past years, the consumer awareness in health and natural foods has increased and led to a movement denominated “Clean Label”. The Clean Label movement avoids using artificial products labeled with E-Numbers, returning to natural foodstuff, paying attention to the packaging and the ingredients in the food. Consequently, the utilization of natural ingredients in the formulation of food and pharmaceutical products has progressively increased. In response to the consumer preference, many products with natural ingredients are being established which has resulted in increasing the market share of these naturally constituted products. In recent years, there has been a growth in natural product developments for a variety of purposes.

Nevertheless, the efforts in formulating all-natural or substantially all-natural nutritional supplements in a solid dosage form contained certified organic natural ingredients have generated intolerable outcomes. In particular, these developments have not lead to reliable characteristics like acceptable tablet hardness, good friability and/or acceptable dissolution profile of the active ingredient. Moreover, the use of natural ingredients so far resulted in adverse processing characteristics, such as reduced flowability, poor compressibility of the powder blend and batch to batch variation of finished product.

In the pharmaceutical and food industries, a number of excipients are used in the manufacture of solid dosage forms. These excipients are widely responsible for the physical characteristics or processing performance of solid dosage forms. The excipients in solid dosage formulations are generally inert substances which deliver the desired dosage and precise administration of active pharmaceutical ingredients. The solid dosage forms known up to date provide multi-functional roles such as improving the patient acceptability and enhancing the bioavailability and/or solubility of the active pharmaceutical ingredients.

Tablets are the most accepted dosage form among pharmaceutical and/or nutritional products. Tablets are manufactured by compressing a suitable powder mixture in a die at high compression force. The powder mixture contains, besides the active pharmaceutical ingredients, usually also fillers, binders, lubricants, glidants and disintegrants. The industrial production of high quality tablets involves a tablet mixture with good properties regarding homogeneity, flowability and compressibility.

There are two main methods to manufacture tablets:

Direct compression: the powder mixture is mixed during a period of time and is then directly compressed into tablets. This method is seen as the most efficient and desirable method. The choice of disintegrants is extremely critical for disintegration of direct compression tablets.
Granulation techniques: consist of wet granulation and dry granulation/slugging methods where binding agents are added in liquid form and in dry form respectively. Some actives exhibit poor fluidity and compressibility and in those case granules with acceptable flow and compression properties have to be formed.

A disintegrant is an excipient in a tablet formulation which is added to the formulation to promote the tablet's disintegration. It assists to break down the tablet into smaller particles such that the surface area is increased when the tablet comes into contact with liquid or fluid matter. In this way, the release of the active nutraceutical and/or pharmaceutical substance is promoted. The function of disintegrant is opposite to the function of the binder. Strong binders thus require strong disintegrants to break down the tablet and release the active nutraceutical and/or pharmaceutical substance.

Super disintegrants are being developed due to the market demands for even faster absorption of the active nutraceutical and/or pharmaceutical substance. Super disintegrants assist in greater effectiveness of the active nutraceutical and/or pharmaceutical substance in lower dosage.

Natural polymers are safe, nontoxic and biocompatible. Therefore, there is a strong demand to use natural polymers as super disintegrant.

To the knowledge of the inventors, up to now there is no described or commercially available ready-to-use natural disintegrant pre-mix in powder form, which exclusively consists of natural polymers, which is suitable for manufacturing tablets, and which shows the properties of a super disintegrant.

Therefore, the object of the present invention is providing ready-to-use natural disintegrant pre-mix in powder form, which exclusively consists of natural polymers, which is suitable for manufacturing tablets, and which shows the properties of a super disintegrant.

Another object of the present invention is to provide an all-natural super disintegrant compound which is prepared by a physical blending procedure without carrying out any chemical reaction process.

### SUMMARY OF THE INVENTION

In one embodiment the present invention relates to a natural disintegrant composition which comprises from about 20% to about 90% by weight of Psyllium Husk and about 5% to about 60% by weight of Oat Fiber (all weight percentages being based on the dry weight of the disintegrant composition).

In another embodiment the present invention relates to a natural disintegrant composition which comprises from about 20% to about 90% by weight of Psyllium Husk; about 5% to about 60% by weight of Oat Fiber; and about 1% to about 25% by weight of at least one further component selected from guar gum, Locust bean gum, gum karaya, Aegle marmelose gum, Mangifera indica gum, and a combination thereof (all weight percentages being based on the dry weight of the disintegrant composition).

In still another embodiment, the present invention relates to a natural disintegrant composition which comprises from about 20% to about 90% by weight of Psyllium Husk; about 5% to about 60% by weight of Oat Fiber; and about 1% to about 20% by weight of at least one further component selected Apple Fiber, Wheat Fiber, Pea Fiber, Cellulose Powder, Agave Powder, Bamboo Fiber, and a combination thereof (all weight percentages being based on the dry weight of the disintegrant composition).

In yet another embodiment the present invention relates to a natural disintegrant composition which consists of from about 20% to about 90% by weight of Psyllium Husk; about 5% to about 60% by weight of Oat Fiber; about 1% to about 25% by weight of at least one further component selected from guar gum, Locust bean gum, gum karaya, Aegle marmelose gum, Mangifera indica gum; and about 1% to about 20% by weight of Apple Fiber, Wheat Fiber, Pea Fiber, Cellulose Powder, Agave Powder, Bamboo Fiber, and a combination thereof (all weight percentages being based on the dry weight of the disintegrant composition).

Psyllium Husk: Psyllium is a soluble fiber obtained from the seeds of Plantago ovata. It is usually found in the form of husk, granules, capsules or powder. The plant contains mucilage in the epidermis of the seeds and has very high percentage of swelling index as compared to other super disintegrating agents. The rapid disintegration is due to the swelling of super disintegrants to create enough hydrodynamic pressure for quick and complete disintegration of the tablet (and capsule “slugs’). The rate of swelling and the significant force of swelling also determine its disintegrating efficiency.

Oat Fiber: Oat Fiber is typically an insoluble fiber made by grinding and purifying the outermost protective layer of the oat grain, composed of lignin, cellulose, and hemicellulose. It is a rich source of insoluble dietary fiber (up to 90%). FDA confirmed oat fiber as dietary fiber under mixed plant cell wall fibers category.

Guar Gum: also called guaran, is a substance made from guar beans, which has thickening and stabilizing properties. The guar seeds are dehusked, milled and screened to obtain the guar gum. It is typically produced as a free-flowing, off-white powder. It is classed as a galactomannan. In Europe, guar gum has EU food additive code E412. The disintegration of the tablet might be due to its instantaneous swelling characteristics when it comes into contact with water and owing to its high hydrophilic nature.

Other natural gum components: Locust bean gum, gum karaya, Aegle marmelose gum, Mangifera indica gum and similar other plant derived gums and mixtures of the foregoing.

Natural Plant Fibers/Dietary Fibers: Apple Fiber, Wheat Fiber, Pea Fiber, Cellulose Powder, Agave Powder, Bamboo Fiber and similar other plant/dietary fibers and mixtures of the foregoing.

Another objective of the present invention is to provide a method for manufacturing an all-natural super disintegrant composition. In an embodiment, the present invention relates to a method for preparing a disintegrant composition according to any of the previous embodiments, wherein the ingredients are mixed by physical blending them without carrying out any chemical reaction process.

### DETAILED DESCRIPTION OF THE INVENTION

In an embodiment of the invention, the natural disintegrant composition pre-mix according to any of the embodiments described herein-above is used in the manufacture of solid dosage forms for oral administration of drugs, vitamins, food supplements. The manufacture of solid dosage forms can be accomplished either by direct compression or granulation techniques.

It has been found by the inventors that the natural disintegrant composition pre-mix according to any of the embodiments described herein-above enhances the disintegration properties of the tablets, if used in direct compression or in granulation procedures. In fact, the natural disintegrant composition provides super disintegrant properties to the tablet, while maintaining excellent properties of homogeneity, flowability and compressibility during the manufacture of the solid oral dosage form. Solid dosage form formulations, such as tablets, made with the disintegrant composition disclosed herein advantageously may be labelled as “natural”. The disclosed disintegrant composition may replace conventional super disintegrants like croscarmellose or crospovidone.

The content of natural disintegrant composition pre-mix according to any of the embodiments described herein-above in the tablet may be about 1 to 10% w/w, preferably between 3 to 5% w/w, based on the total weight of the tablet. Natural excipients are preferably used in the pharmaceutical and food industry because of their biocompatibility, non-toxicity, low cost and renewable resources for a sustainable supply. Furthermore, the tablet formulated with an all-natural disintegrant composition pre-mix has a great acceptability by the consumers, which is due to utilization of clean labeled ingredients.

The quantity of the Psyllium Husk, which is the main disintegrant in the premix, is within the range about 20% to about 90% by weight of the dry disintegrant composition. The grade of the Psyllium Husk can be organic and/or according to EP and USP/NF. A range of about 30% to 70% by weight of the dry disintegrant composition is preferred.

The quantity of secondary disintegrant, Oat Fiber, in the premix is within the range of about 5% to about 60% by weight of the dry disintegrant composition. The grade of the Oat Fiber can be organic certified. A range of 20% to 40% by weight of Oat Fiber in the dry disintegrant composition is preferred.

A plant fiber/dietary fiber like Bamboo Fiber or Agave Powder can be added into the disintegrant composition within a range of 1% to about 20% by weight of the dry disintegrant composition. The plant fiber/dietary fiber improves the manufacturing of the pre-mix. The grade of the fiber can be organic certified or conventional. A range of 1% to 8% by weight of the dry disintegrant composition is preferred.

A gum component such as guar gum can alternatively or additionally be added to the premix within the range of 1% to about 25% by weight of the dry disintegrant composition. The grade of guar gum can be organic certified or conventional. A range of 1% to 15% by weight of the dry disintegrant composition is preferred.

Hereinafter, the present invention will now be described in detail with reference to the appended examples. It is to be understood, however, that these examples are for illustrative purposes only and are not intended to limit the scope of the present invention.

### EXAMPLES

The following examples 1 to 5 illustrate natural disintegrant compositions according to present invention. All percentages are by weight.

### Example 1

A dry premix composition is produced by simple mixing of the ingredients as set out in Table 1 in a dry powder mill/blender, such as a V-type powder blender until a homogenous mixture is achieved, resulting in a formulation of the inventive disintegrant premix composition for manufacturing solid dosage forms. Optionally the individual ingredient or the dry powder blend may also be milled to form a fine powder to enhance the functionality and performance of the super disintegrant blend in solid oral dosage forms such as tablets (and capsule ‘slugs’).

TABLE 1

| Excipient: | Grades: | % W/W |
|---|---|---|
| Psyllium Husk | USP/NF/o-Organic/Conventional | 60 |
| Oat Fiber | Organic/Conventional | 37 |
| Bamboo Fiber | Organic/Conventional | 1.9 |
| Agave Powder | Organic/Conventional | 1.1 |

### Examples 2 to 5

In the following examples, the dry disintegrant premix compositions were produced as in example 1, however with different ingredients:

### Example 2

TABLE 2

| Excipient: | Grades: | % W/W |
|---|---|---|
| Psyllium Husk | USP/NF/Organic/Conventional | 50 |
| Oat Fiber | Organic/Conventional | 30 |
| Guar Gum | Organic/Conventional | 15 |
| Agave Powder | Organic/Conventional | 5 |

### Example 3

TABLE 3

| Excipient: | Grades: | % W/W |
|---|---|---|
| Psyllium Husk | USP/NF/Organic/Conventional | 60 |
| Oat Fiber | Organic/Conventional | 35 |
| Bamboo Fiber | Organic/Conventional | 1.9 |
| Pea Fiber | Organic/Conventional | 2.1 |

### Example 4

TABLE 4

| Excipient: | Grades: | % W/W |
|---|---|---|
| Psyllium Husk | USP/NF/Organic/Conventional | 60 |
| Oat Fiber | Organic/Conventional | 30 |
| Bamboo Fiber | Organic/Conventional | 5 |
| Wheat Fiber | Organic/Conventional | 5 |

### Example 5

TABLE 5

| Excipient: | Grades: | % W/W |
|---|---|---|
| Psyllium Husk | USP/NF/Organic/Conventional | 60 |
| Oat Fiber | Organic/Conventional | 30 |
| Bamboo Fiber | Organic/Conventional | 5 |
| Apple Fiber | Organic/Conventional | 5 |

### Example 6—Results and Discussion

The present invention was developed to ensure efficient tablet disintegration and batch to batch consistency.

According to this, a test series was defined to validate the influence of the invention on tablet disintegration time. Therefore, several formulations with different disintegrant contents and different active nutraceutical/pharmaceutical substances (Calcium, Vitamin E, L-Arginine) were investigated due to their disintegration time.

Consequently, tablets were compressed with a standard round tooling with 10 mm diameter, 5 mm thickness and engraving. For the characterization, the tablets were tested on disintegration time (according to Ph. Eur. 2.9.1).

The preferred formulas for the testing of super disintegrant premix composition are set out in the following table:

TABLE 6

| | | Formulation I | Formulation II |
|---|---|---|---|
| Excipient | Grades | (% w/w) | (% w/w) |
| Psyllium Husk | USP/NF/organic | 60 | 50 |
| Oat Fiber | organic | 37 | 30 |
| Guar Gum | organic | N/A | 15 |
| Bamboo Fiber | organic | 1.9 | N/A |
| Agave Powder | organic | 1.1 | 5 |

The following table illustrates the used formulations and the results. All percentages are by weight. Croscarmellose were used as reference super disintegrant.

| | | disintegration time | | | | | | | | | | | | |
|---|---|---|---|---|---|---|---|---|---|---|---|---|---|---|
| | | | | | | | +5% | | +3% | | +5% | | +3% | |
| | | no | +3% | | +5% | | formulation | | formulation | | formulation | | formulation | |
| Tablet formula | % | disintegrants | crosscarm. | | crosscarm. | | 1 | | 1 | | 2 | | 2 | |
| MCC 200 | 65.55 | 28:13 min | 6:21 | min | 4:07 | min | 1:30 | min | 1:43 | min | 1:50 | min | 2:30 | min |
| Dicafos A150 | 28.5 | 29:56 min | 7:05 | min | 4:33 | min | 1:25 | min | 1:50 | min | 1:50 | min | 2:30 | min |
| HPC | 2.7 | 35:42 min | 8:11 | min | 5:11 | min | 2:20 | min | 2:03 | min | 2:05 | min | 2:57 | min |
| MCC 105 | 1.8 | | | | | | | | | | | | | |
| Silicon dioxide | 0.5 | | | | | | | | | | | | | |
| aerosil | | | | | | | | | | | | | | |
| Magnesiumstearat | 0.95 | | | | | | | | | | | | | |
| Vitamin E | 30 | 39:12 min | 13:12 | min | 11:30 | min | 22:40 | min | 23 | min | 21:50 | min | 24 | min |
| Riboflavin | 30 | 39:28 min | 12:56 | min | 11:31 | min | 24 | min | 23 | min | 23 | min | 24 | min |
| MCC 200 | 20 | 40:16 min | 13:22 | min | 11:40 | min | 24 | min | 24:30 | min | 23 | min | 25 | min |
| Dicafos A150 | 19 | | | | | | | | | | | | | |
| Magnesiumstearat | 1 | | | | | | | | | | | | | |
| L-Arginin | 60 | 15:16 min | 7:12 | min | 5:40 | min | 9:00 | min | 9:17 | min | 8:33 | min | 9:09 | min |
| MCC 200 | 20 | 16:10 min | 6:59 | min | 6:00 | min | 9:57 | min | 9:20 | min | 8:58 | min | 9:27 | min |
| Dicafos A150 | 19 | 18:34 min | 7:02 | min | 6:15 | min | 10:12 | min | 10:40 | min | 9:00 | min | 10:20 | min |
| Magnesiumstearat | 1 | | | | | | | | | | | | | |
| Dicafos A150 | 90.25 | intact till | less than | | less than | | 1:00 | min | 0:56 | min | 1:02 | min | 0:54 | min |
| MCC 200 | 7 | 60 mins | 1 min | | 1 min | | 1:00 | min | 0:57 | min | 1:02 | min | 0:54 | min |
| Stearic acid | 2 | | | | | | 1:01 | min | 0:57 | min | 1:01 | min | 0:55 | min |
| Silicon dioxide | 0.25 | | | | | | | | | | | | | |
| aerosil | | | | | | | | | | | | | | |
| Magnesiumstearat | 0.5 | | | | | | | | | | | | | |

Based on the results of the tablet disintegration time comparison it can be inferred that the invention has a positive influence on tablet disintegration which is comparable and in times even enhanced to the non-natural croscarmellose. Furthermore, the results show that already a small content of the premix-composition according to the present invention, of 3% w/w in the tablet formulation results in acceptable disintegration values which are improved to tablets containing no disintegrant or a known non-natural super disintegrant like croscarmellose. Due to this fact, the invention may advantageously be used as an excellent natural/organic super disintegrant with good properties and as potential substitute for conventional synthetic or not all natural super disintegrants.

1. A natural disintegrant composition which comprises from about 30% to about 90% by weight of Psyllium Husk, about 20% to about 40% by weight of Oat Fiber, and about 1% to about 25% by weight of at least one further component selected from the group consisting of guar gum, Locust bean gum, gum karaya, Aegle marmelose gum, Mangifera indica gum, and a combination thereof (all weight percentages being based on the dry weight of the disintegrant composition).

2. The natural disintegrant composition of claim 1, further comprising about 1% to about 20% by weight of at least one further component selected from the group consisting of Apple Fiber, Wheat Fiber, Pea Fiber, Cellulose Powder, Agave Powder, Bamboo Fiber, and a combination thereof (all weight percentages being based on the dry weight of the disintegrant composition).

3. The natural disintegrant composition of claim 1, wherein the component selected from the group consisting of Apple Fiber, Wheat Fiber, Pea Fiber, Cellulose Powder, Agave Powder, Bamboo Fiber, and a combination thereof is present in an amount ranging from about 1% to about 8% by dry weight of the composition.

4. The natural disintegrant composition of claim 1, wherein the component selected from the group consisting of guar gum, Locust bean gum, gum karaya, Aegle marmelose gum, Mangifera indica gum, and a combination thereof is present in an amount ranging from about 1% to about 15% by dry weight of the composition.

5. A method for producing a natural super disintegrant composition of claim 1, wherein a mixture comprising about 30% to about 90% by weight of Psyllium Husk, about 20% to about 40% by weight of Oat Fiber, and about 1% to about 25% by weight of at least one further component selected from the group consisting of guar gum, Locust bean gum, gum karaya, Aegle marmelose gum, Mangifera indica gum, and a combination thereof (all weight percentages being based on the dry weight of the disintegrant composition), is provided and physically blended until a homogenous mixture is attained.

6. A method for producing a natural super disintegrant composition of claim 2, wherein a mixture comprising about 30% to about 90% by weight of Psyllium Husk, about 20% to about 40% by weight of Oat Fiber, about 1% to about 25% by weight of at least one further component selected from the group consisting of guar gum, Locust bean gum, gum karaya, Aegle marmelose gum, Mangifera indica gum, and a combination thereof, and about 1% to about 20% by weight of at least one further component selected the group consisting of Apple Fiber, Wheat Fiber, Pea Fiber, Cellulose Powder, Agave Powder, Bamboo Fiber, and a combination thereof (all weight percentages being based on the dry weight of the disintegrant composition), is provided and physically blended until a homogenous mixture is attained.

7. A method for producing a solid dosage form formulation comprising forming a solid dosage form from the natural disintegrant composition of claim 1.

8. The method of claim 7, wherein the solid dosage form formulation is a tablet.

9. A solid dosage form formulation comprising the disintegrant composition of claim 1 in an amount ranging from about 1% to about 10%, based on the total weight of the solid dosage form formulation on a dry weight basis.

10. The solid dosage form formulation of claim 9, comprising the disintegrant composition in an amount ranging from about 3% to about 5% w/w, based on the total weight of the tablet in the tablet formulation on a dry weight basis.

11. The solid dosage form formulation of claim 9, which is in the form of a tablet.
